(19) United States
(12) Patent Application Publication
(10) Pub. No.: US 2024/0150821 A1
(43) Pub. Date: May 9, 2024

(54) METHODS OF ANALYZING A BIOLOGICAL SAMPLE

(71) Applicant: Wallac Oy, Turku (FI)

(72) Inventors: Ville Veikkolainen, Turku (FI); Mikko Aaltoranta, Turku (FI)

(21) Appl. No.: 17/773,273

(22) PCT Filed: Oct. 26, 2021
(86) PCT No.: PCT/IB2021/059896

§ 371 Date: Apr. 29, 2022

Related U.S. Application Data
(60) Provisional application No. 63/106,094, filed on Oct. 27, 2020.

(51) Int. Cl. C12Q 1/6851

(52) U.S. Cl. CPC C12Q 1/6851

(57) ABSTRACT

The present disclosure relates to a method, system, and kit for analyzing a biological sample that has been dried on a sample carrier, the method comprising: a) creating a combination within a vessel, the combination comprising the sample carrier, an elution solution, and a reaction solution, wherein the sample carrier has not been subjected to incubation prior to creating the combination; and b) incubating the combination within the vessel to elute the biological sample from the sample carrier and to perform qPCR on the biological sample.

### CROSS-REFERENCE

This application claims priority to U.S. Provisional Patent Application No. 63/106,094, titled “Methods of Analyzing a Biological Sample,” filed on Oct. 27, 2021, which is incorporated herein by reference in its entirety.

### BACKGROUND

Biological samples can be used for multiple types of analytical testing. Biological samples such as blood contain DNA which can be analyzed through analytical testing methods such as polymerase chain reaction (PCR). Dried blood spot (DBS) testing is a form of biological sampling where blood samples are blotted and dried on paper. The dried samples can be shipped to a laboratory and analyzed using various methods. The DBS contains DNA which can be extracted using a variety of chemical and/or physical methods. A DNA extraction is often used to remove cell debris and other insoluble material that can interfere with subsequent analysis. The eluted DNA can then be used for a variety of tests (e.g., PCR). DNA extraction from samples can be time-consuming.

### SUMMARY

Provided herein are technologies, systems, apparatuses, and methods of analyzing a biological sample, where at least some of the methods comprise, a) obtaining a sample carrier that comprises the biological sample therein, b) creating a combination within a vessel comprising the sample carrier, an elution solution, and a reaction solution, wherein the sample carrier has not been subjected to incubation prior to creating the combination; and, c) incubating the combination within the vessel to elute the biological sample from the sample carrier and to perform qPCR on the biological sample. As used herein, references to elution of the biological sample from the sample carrier may include partial or complete elution, and elution need only be performed to the extent necessary to allow for the analysis of the biological sample in accordance with the disclosed methods and systems.

The biological sample may be a blood sample, such as, for example, a liquid whole blood sample, components of whole blood, such as, but not limited to, red blood cells, white blood cells, or plasma, or components of whole blood or whole blood in combination with other substances. The blood sample may be in the form of a dried blood spot (DBS). In some DBS embodiments, the DBS may be about 0.5 mm to about 5.0 mm in diameter. In some DBS embodiments, the solution and the DBS sample remain in the vessel while performing the qPCR.

The biological sample may be a saliva sample, such as, for example, a liquid saliva sample, and/or, for example, a dried saliva spot (DSS).

In some embodiments, the biological sample may be a buccal sample, and may be a dried buccal sample. The solution and the dried buccal sample remain in the vessel while performing the qPCR.

The disclosed methods do not include a washing prior to c). Prior to b) the one or more qPCR reagents may be lyophilized in the vessel. Prior to b), the vessel comprising the one or more lyophilized qPCR reagents may be stored at a temperature of about −20° C. to about 35° C.

In some embodiments, the incubation is about 5 minutes to about 60 minutes. The vessel may be incubated at a temperature of about 20° C. to about 100° C., while in some instances, the vessel may be incubated at about 95° C. In some embodiments, the vessel is incubated less than 5 minutes.

In some embodiments, the one or more qPCR reagents comprise a polymerase, and may include a Taq polymerase. In some embodiments, the one or more qPCR reagents include a deoxyuridine triphosphate (dUTP). In embodiments, the one or more qPCR reagents may comprise one or more dNTPs and/or magnesium chloride. The one or more qPCR reagents may comprise one or more inhibitor resistant polymerases.

The one or more qPCR reagents may comprise one or more primers and/or one or more probes. In embodiments, the one or more of the probes may include a fluorescent probe. The one or more qPCR reagents may include a preservative, such as, for example, a biocide.

The one or more qPCR reagents may comprise one or more salts in certain embodiments, and the one or more salts may be selected from a group comprising and/or consisting of NH₄Cl, KCl, MgCl₂, Mg²⁺, K⁺, and combinations thereof. In embodiments, the one or more salts is Mg′ at a concentration of about 0.5 mM to about 5.0 mM. In some embodiments, the one or more salts is K⁺ at a concentration of about 35 mM to about 100 mM.

The one or more qPCR reagents may include, for example, dimethylsulfoxide (DMSO), and at a concentration of about 1% to about 10%. The one or more qPCR reagents may comprise formamide at a concentration of about 1% to about 10%.

The one or more qPCR reagents may comprise bovine serum albumin, for example, at a concentration of about 10 μg/ml to about 100 μg/ml, and/or Betaine, for example, at a concentration of about 0.5 M to about 2.5 M. In some embodiments, the one or more qPCR reagents include an antibody.

The one or more qPCR reagents may include an indicator, where such indicator may be, e.g., selected from a group comprising and/or consisting of cresol red, bromophenol blue, and xylene cyanol. In embodiments, the indicator is cresol red.

The one or more qPCR reagents may include a stabilizer, and the stabilizer may be e.g., selected from a group comprising or consisting of trehalose, mannitol, bovine serum albumin (BSA), polyethylene, and combinations thereof. In some embodiments, the stabilizer is lyophilized. In embodiments, the qPCR may be performed without a pre-amplification.

In some embodiments, the incubating further comprises incubating at a first temperature to elute the biological sample, and incubating at a second temperature to perform qPCR. In some embodiments, the first temperature is about 20° C. to about 100° C. In some embodiments, the first temperature is about 95° C. In some embodiments, the incubating at the first temperature is about 5 minutes to about 60 minutes. In some embodiments, incubating at the second temperature to perform qPCR further comprises incubating in accordance with a qPCR protocol.

In some embodiments, the biological sample is blood. In some embodiments, the blood is liquid blood. In some embodiments, the blood is a dried blood spot (DBS). In some embodiments, the DBS is about 0.5 mm to about 5.0 mm in diameter. In some embodiments, the elution solution and the DBS are not removed from the vessel prior to performing the qPCR. In some embodiments, the biological sample is saliva. In some embodiments, the saliva is liquid saliva. In some embodiments, the saliva is a dried saliva spot (DSS). In some embodiments, the biological sample is collected on a swab. In some embodiments, the biological sample collected on the swab is selected from the group consisting of buccal cells, nasal tissue and/or mucosa, throat tissue and/or mucosa, and combinations thereof. In some embodiments, the biological sample is collected on the swab from a surface. In some embodiments, the biological sample collected on the swab is dried.

In some embodiments, a) and b) do not include a washing. In some embodiments, prior to a) the reaction solution is lyophilized in the vessel. In some embodiments, prior to a), the vessel comprising the lyophilized reaction solution is stored at a temperature of about −20° C. to about 35° C.

In some embodiments, the reaction solution comprises a polymerase. In some embodiments, the polymerase is an inhibitor resistant polymerase. In some embodiments, the polymerase is Taq polymerase. In some embodiments, the reaction solution includes deoxyuridine triphosphate (dUTP). In some embodiments, the reaction solution comprises one or more dNTPs.

In some embodiments, the reaction solution comprises magnesium chloride. In some embodiments, the reaction solution comprises one or more primers. In some embodiments, the reaction solution comprises one or more probes. In some embodiments, one or more of the probes is a fluorescent probe. In some embodiments, the reaction solution comprises a preservative. In some embodiments, the preservative is a biocide. In some embodiments, the reaction solution comprises one or more salts. In some embodiments, the one or more salts is selected from the group consisting of NH₄Cl, KCl, MgCl₂, Mg²⁺, K⁺, and combinations thereof. In some embodiments, the one or more salts is Mg²⁺ at a concentration of about 0.5 mM to about 5.0 mM. In some embodiments, the one or more salts is K⁺ at a concentration of about 35 mM to about 100 mM.

In some embodiments, the reaction solution includes dimethylsulfoxide (DMSO) at a concentration of about 1% to about 10%. In some embodiments, the reaction solution comprises formamide at a concentration of about 1% to about 10%. In some embodiments, the reaction solution comprises bovine serum albumin at a concentration of about 10 μg/ml to about 100 μg/ml. In some embodiments, the reaction solution comprises Betaine at a concentration of about 0.5 M to about 2.5 M. In some embodiments, the reaction solution includes an antibody. In some embodiments, the reaction solution includes an indicator. In some embodiments, the indicator is selected from the group consisting of cresol red, bromophenol blue, and xylene cyanol. In some embodiments, the indicator is cresol red. In some embodiments, the reaction solution includes a stabilizer. In some embodiments, the stabilizer is selected from the group consisting of trehalose, mannitol, bovine serum albumin (BSA), polyethylene, and combinations thereof. In some embodiments, the stabilizer is lyophilized. In some embodiments, the qPCR is performed without a pre-amplification. In some embodiments, the sample carrier is at least a portion of a dried blood spot card.

Also disclosed are methods of analyzing a biological sample, the methods comprising: a) obtaining a sample carrier that comprises the biological sample dried thereon; b) incubating the sample carrier with an elution solution to elute the biological sample from the sample carrier; and c) contacting a vessel comprising a reaction reagent dried thereon with the eluted biological sample to perform qPCR on the eluted biological sample.

In some embodiments, the reaction reagent is dried for about 30 to about 40 minutes on a surface of the vessel. In some embodiments, the reaction reagent is dried at a temperature ranging from about 75° C. to about 85° C. on a surface of the vessel. In some embodiments, the elution solution for about 15 minutes to about 25 minutes. In some embodiments, the sample carrier is incubated with the elution solution at a first temperature. In some embodiments, the first temperature is about 65° C. to about 75° C. In some embodiments, step b) further comprises subjecting the sample carrier and elution solution to a shaking motion at a speed of about 600 revolutions per minute (RPM) to about 800 RPM.

Also disclosed are systems for analyzing a biological sample dried on a sample carrier, where the systems include a vessel; a liquid handler; a qPCR system configured to perform qPCR on the biological sample; and a controller configured to cause the liquid handler to create a combination in the vessel, the combination including an elution solution, a reaction solution, and the biological sample dried on the sample carrier, where the sample carrier has not been subjected to incubation prior to creating the combination. The system may further include a biological sample puncher configured to extract a portion of the sample carrier that comprises at least a portion of the dried biological sample. In embodiments, the vessel is or includes a microtiter plate. In some embodiments of the disclosed systems, the biological sample is a dried blood spot and the biological sample puncher is a dried blood spot puncher.

Disclosed herein are kits for use in analyzing a biological sample dried on a sample carrier, where the kit includes an elution solution; a reaction solution; and instructions for performing qPCR on a biological sample dried on a sample carrier, the instructions including: (i) creating a combination, the combination comprising the sample carrier with the biological sample dried thereon, the reaction solution, and the elution solution, wherein the sample carrier has not been subjected to incubation prior to creating the combination; and (ii) incubating the combination to elute the biological sample carrier and to perform qPCR on the biological sample, wherein said instructions do not include instructions for subjecting the sample carrier with the biological sample dried thereon to incubation prior to creating the combination. In embodiments, the instructions include instructions to avoid and/or not subject the sample carrier with the biological sample dried thereon to incubation prior to creating the combination. In embodiments, the kit also includes instructions to create the combination in a vessel, including for example, embodiments wherein the vessel is a microtiter plate.

Some embodiments of the methods, systems, and kits described herein may provide one or more of the following advantages.

First, some embodiments described herein may provide a simple and rapid qPCR assay. For example, in some embodiments, the methods, systems, and kits provided herein require fewer steps to be performed by a user while providing quick results (e.g., in less than about 110 minutes). In some embodiments, the fewer steps can include combining a sample carrier, an elution solution, and a reaction solution and incubating this combination within a vessel to elute a biological sample from the sample carrier and perform qPCR on the biological sample. In some embodiments, the fewer steps can include incubating the sample carrier with an elution solution to elute the biological sample from the sample carrier and contacting a vessel comprising a reaction solution dried thereon with the eluted biological sample to perform qPCR on the eluted biological sample. In some embodiments, the methods, systems, and kits do not require any washing and thus, may minimize the hands-on work of the user, reduce unnecessary biohazardous waste, and reduce potential sources of sample contamination. In some embodiments, the exclusion of washing may also diminish the possibility that any target DNA is washed away.

Second, some embodiments described herein may provide efficient qPCR workflows. For example, in some embodiments, the methods, systems, and kits described herein may reduce the need for instrumentation, thereby enabling a more streamlined and/or automated workflow. In some embodiments, the methods, systems, and kits described herein may require less consumables and pipetting, thereby yielding a faster turnaround time and less variation in results as compared to conventional qPCR workflows. In some embodiments, the methods, systems, and kits described herein may reduce the incidence of technical errors and/or sample contamination as compared to conventional qPCR workflows.

Third, some embodiments described herein may provide cost-effective qPCR workflows. For example, in some embodiments, the methods, systems, and kits described herein may include reagents that are stable and can be stored and shipped in ambient temperature. Furthermore, in some embodiments, the methods, systems, and kits of the disclosure may not require the user to use a clean room as there may be no need for the user to handle any qPCR reagents (e.g., when the vessel provided comprises the reaction solution dried thereon).

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### DESCRIPTION OF THE DRAWINGS

The following drawings illustrate certain embodiments of the features and advantages of this disclosure. These embodiments are not intended to limit the scope of the appended claims in any manner. Like reference symbols in the drawings indicate like elements.

FIG. 1 shows an example workflow for a one-step extraction and qPCR reaction.

FIG. 2A depicts a comparison of the Ct values of a four-analyte multiplex qPCR assay performed on dried blood spot discs or whole adult blood with NeoMDx™ PCR master mix and a mixture of plasmid (RPP30 Cy5.5, SMN1 Cy5, and TREC FAM) and other (CMV HEX in adult whole blood) PCR targets. qPCR assays were performed on plasmids, without DNA as a negative control (Blank), on control extracted DNA on dried blood spots (C3 in situ), or on adult whole blood spiked with Cytomegalovirus (CMV8 in situ).

FIG. 2B depicts a comparison of the Ct values of a four-analyte multiplex qPCR assay performed on dried blood spot discs with NeoMDx™ PCR master mix with some components replaced by TaqPath™ ProAmp™ master mix. Four analytes are KREC, RPP30, SMN1, and TREC plasmids.

FIG. 2C depicts a comparison of the Ct values of a four-analyte multiplex qPCR assay performed on dried blood spots with NeoMDx™ PCR Master Mix. The four analytes include KREC, RPP30, SMN1, and TREC plasmids.

FIG. 3A depicts a comparison of the Ct values of qPCR assays performed on dried blood spots or purified plasmid DNA template with NeoMDx™ enzyme with a combination of KREC, RPP30 Cy5.5, SMN1 Cy5, or TREC Fam plasmid as a DNA template.

FIG. 3B depicts a comparison of the Ct values of qPCR assays performed on dried blood spots or purified plasmid DNA template with TaqPath™ enzyme with a combination of KREC, RPP30 Cy5.5, SMN1 Cy5, or TREC Fam plasmid as DNA template.

FIG. 4A depicts a comparison of the Ct values of a four-analyte multiplex qPCR assay performed on dried blood spot discs with dry chemistry. Four analytes are KREC, RPP30. SMN1, and TREC plasmids.

FIG. 4B depicts a comparison of the Ct values of a four-analyte multiplex qPCR assay performed on dried blood spot discs and fresh PCR mix as a control for the dry chemistry experiment. Four analytes are KREC, RPP30. SMN1, and TREC plasmids.

FIG. 5 discloses one embodiment of a system as disclosed herein.

FIG. 6 shows an example workflow, including the duration of each step, of an inhibitor-tolerant dry chemistry qPCR assay with a one-step DNA extraction from dry blood spots (DBSs).

FIG. 7 depicts a comparison between a conventional wet chemistry and dry chemistry qPCR assays. The DBS samples for the wet chemistry were processed with a multistep extraction protocol resulting in a much more purified DNA sample. The dry chemistry DBS samples were extracted using the disclosed protocol resulting in a crude DNA sample with high concentrations of blood-based inhibitors.

### DETAILED DESCRIPTION

### I. Definitions

In order that the present disclosure can be more readily understood, certain terms are first defined. As used in this application, except as otherwise expressly provided herein, each of the following terms shall have the meaning set forth below. Additional definitions are set forth throughout the application.

Unless defined otherwise, all technical and scientific terms used herein have the same meaning as commonly understood by one of ordinary skill in the art to which this disclosure belongs. Each of the patents, applications, published applications, and other publications that are mentioned throughout the specification and the attached appendices are incorporated herein by reference in their entireties. Units, prefixes, and symbols are denoted in their Systéme International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that aspects and embodiments of the disclosure described herein include “comprising,” “consisting,” and “consisting essentially of” aspects and embodiments. It is understood that aspects and variations of the embodiments described herein include “consisting of” and/or “consisting essentially of” aspects and variations. In some embodiments, the methods and compositions of the present invention can comprise, consist of, or consist essentially of the listed steps. As used herein the term “consisting essentially of” shall be construed to mean including the steps and such additional ingredients or steps which do not materially affect the basic and novel properties of the method. In some embodiments, a method in accordance with embodiments of the present invention that “consists essentially of” the recited steps does not include any additional steps that alter the basic and novel properties of the method (e.g., the elution of DNA absent a washing step, performing qPCR absent a pre-amplification step, performing qPCR without removing the biological sample).

The terms “about” and “approximately” as used herein shall generally mean an acceptable degree of error for the quantity measured given the nature or precision of the measurements. Typical, exemplary degrees of error are within 20 percent (%), preferably within 10%, and more preferably within 5% of a given value or range of values. Any reference to “about X” specifically indicates at least the values X, 0.95X, 0.96X, 0.97X, 0.98X, 0.99X, 1.01X, 1.02X, 1.03X, 1.04X, and 1.05X. Thus, “about X” is intended to teach and provide written description support for a claim limitation of, e.g., “0.98X.” The terms “about” and “approximately,” particularly in reference to a given quantity, encompass and describe the given quantity itself.

Alternatively, in biological systems, the terms “about” and “approximately” may mean values that are within an order of magnitude, preferably within 5-fold, and more preferably within 2-fold of a given value. Numerical quantities given herein are approximate unless stated otherwise, meaning that the term “about” or “approximately” can be inferred when not expressly stated.

As used herein the term “vessel” refers to a container used for the containment of a solid, a liquid, or a gas. A vessel can be used to contain a sample, reagents, and/or solutions for analytical testing. In some embodiments, a vessel can contain a blood sample, PCR reagents (e.g., qPCR reagents), and solutions for the analytical testing. Non-limiting examples of vessels can include plastic or glass tubes. In some embodiments, the vessel can have a conical shape. In some embodiments, the vessel(s) be tubes that are coupled together in a strip. In other examples, the vessel(s) can be coupled together in a tray (e.g., a 96-well format). Vessels can include a lid that can be attached to a conical portion of the vessel, or separate from the conical portion of the vessel. In some embodiments, a vessel can be used without a lid but instead use a film, foil, and/or mineral oil to prevent the evaporation of the sample, reagents, and/or solutions within the vessel.

As described herein, any concentration range, percentage range, ratio range, or integer range is to be understood to include the value of any integer within the recited range and, when appropriate, fractions thereof (such as one tenth and one hundredth of an integer), unless otherwise indicated.

Analytical testing can be performed on a sample obtained from a subject to determine one or more conditions related to the subject. Biological samples that contain DNA are often targeted for analytical testing. DNA is eluted from the biological sample before the biological sample can be used for analytical testing such as PCR (e.g., qPCR). The current process of eluting DNA from a biological sample can include one or more washing steps. Washing steps can be time-consuming and technically difficult. Wash steps can complicate automated sample processing and increase the time to process samples.

In some examples, a biological sample can be dried on a sample carrier for the ease of sample collection and transport. In examples, a sample carrier is paper or filter paper. For example, biological samples dried in spots on paper can facilitate easy storage and shipping of the sample. Using a sample carrier such as paper for the collection and transfer of biological samples is a popular technique used to collect biological samples. However, problems associated with the elution of DNA from a dried biological sample are similar to those discussed herein and can require one or more washing steps to produce a sample (e.g., DNA sample) sufficient for analytical testing. Multi-step processes to elute DNA from a non-dried or dried biological sample can be slow, costly, and require training and expertise. There is a need for a cost-effective, fast, and user-friendly solutions to the processing of biological samples for analytical testing.

Disclosed herein are methods, systems, and kits to elute DNA from a biological sample and perform analytical tests (e.g., without washing procedures and/or pre-amplification procedures). Embodiments herein describe analyzing a biological sample that has been dried on a sample carrier, the method comprising: a) creating a combination within a vessel, the combination comprising the sample carrier, an elution solution, and a reaction solution, wherein the sample carrier has not been subjected to incubation prior to creating the combination; and b) incubating the combination within the vessel to elute the biological sample from the sample carrier and to perform qPCR on the biological sample.

As described herein, a combination refers to combining the biological sample (e.g., on a sample carrier), an elution solution to elute DNA from the biological sample, and a reaction solution comprising one or more PCR reagents. In some examples, the one or more PCR reagents may be qPCR reagents to be used in connection with a qPCR reaction. In some examples, the elution solution comprises a ratio (e.g., 1:1) of a base (e.g., KOH) and buffer (e.g., Tris buffer).

As described herein, the biological sample can be cell culture, cell biopsy, buccal cells, blood, plasma, hair, urine, saliva, feces, extracted DNA, and extracted RNA. DNA can be eluted from the biological sample within the vessel and used as a template for the qPCR reaction. In some embodiments, the biological sample and the sample carrier remains in the vessel for, and/or is not removed from the vessel prior to, the qPCR reaction.

For example, as described herein, a combination can be created within a vessel, the combination comprising: a biological sample, an elution solution and a reaction solution. The biological sample, an elution solution, and a reaction solution comprising one or more qPCR reagents can be added to the vessel with the biological sample. For example, a biological sample can be dried on a sample carrier and added to a vessel with an elution solution to elute DNA form the biological sample, and a reaction solution. The elution solution can include a buffer and a base. In some examples, the base is KOH and the buffer is Tris buffer.

In some embodiments, the reaction solution comprises one or more qPCR reagents. For example, the reaction solution can include one or more qPCR reagents such as dNTPS, MgCl₂, an inhibitor resistant polymerase suitable for PCR or qPCR, probes and or primers, and an elution solution comprising about 30 mM Tris and about 20 mM KOH where the reaction solution and elution solution are at about a 1:1 ratio. In this example, DNA can be eluted from the biological sample via an incubation in the vessel with the solution comprising one or more qPCR reagents. In some embodiments, the qPCR reaction is performed without pre-amplification. Said differently, in some embodiments, the solution comprising an elution solution, a reaction solution (e.g., including one or more qPCR reagents) and the biological sample on a sample carrier are not removed from the vessel prior to performing the qPCR reaction.

In some embodiments, after the combination including the biological sample on a sample carrier, the elution solution, and the reaction solution comprising one or more qPCR reagents are incubated in the vessel (to facilitate DNA elution), the qPCR reaction can be performed in the same vessel (e.g., in situ). In some embodiments, the DNA is eluted (as described herein) from the biological sample and qPCR is performed within the vessel absent a washing step. In such embodiments, the biological sample may be a dried biological sample on a sample carrier (e.g., a piece of paper) and the dried biological sample and the paper can remain in the vessel with the elution solution and the reaction solution including one or more qPCR reagents for the qPCR reaction.

In some embodiments, prior to a), the reaction solution comprising one or more qPCR reagents are lyophilized in the vessel. The vessel with the lyophilized qPCR reagents may be stored for a period of time prior to the addition of the biological sample. The one or more qPCR reagents may comprise a stabilizer, that may comprise, and/or be selected from the group consisting of trehalose, mannitol, bovine serum albumin (BSA), polyethylene, and combinations thereof. In embodiments, the elution solution may reconstitute the lyophilized reaction solution in the vessel. The vessel including the biological sample on a sample carrier, the elution solution, and the reaction solution comprising the one or more qPCR reagents may be incubated for a period of time, e.g., less than 5 minutes, prior to the start of the qPCR reaction. In some embodiments, a) and b) are performed without a wash step, and/or the qPCR reaction may be performed without a pre-amplification step. Said differently, in some embodiments, the elution solution, and the reaction solution comprising one or more qPCR reagents and the sample carrier including the biological sample may remain in the vessel for the qPCR reaction and/or are not removed from the vessel prior to performing the qPCR reaction.

Disclosed herein, in certain embodiments, are methods of analyzing a biological sample, the methods comprising a) obtaining a sample carrier that comprises the biological sample dried thereon; b) incubating the sample carrier with an elution solution to elute the biological sample from the sample carrier; and c) contacting a vessel comprising a reaction solution dried thereon with the eluted biological sample to perform qPCR on the eluted biological sample.

In some embodiments, qPCR can utilize fluorescent probes for detection. A biological sample such as a blood sample can introduce heme protein into the qPCR reaction. Heme protein can be an inhibitor to qPCR. Heme protein can have a broad florescent spectrum which can cause interference in the detection of a signal coming from qPCR probes used in an in situ qPCR reaction. Performing qPCR of DNA eluted from a DBS sample can be difficult due to the inhibitors present in the biological sample. For example, eluting DNA from a DBS sample can introduce inhibitors such as heme protein when the DNA is eluted in situ. The inhibitors can be overcome by the use of a polymerase and/or master mixes configured for qPCR.

Embodiments herein describe a method of analyzing a biological sample. Analyzing a biological sample can include performing polymerase chain reaction (PCR) on the biological sample. DNA can be eluted and/or extracted from the biological sample and can be used as a template for PCR. Non-limiting examples of PCR include amplified fragment length polymorphism (AFLP) PCR, allele specific PCR (AS-PCR), Alu-PCR, assembly PCR, asymmetric PCR, co-amplification at lower denaturation temperature-based PCR (COLD-PCR), colony PCR, conventional PCR, digital PCR (dPCR), fast cycling PCR, high-fidelity PCR, high-resolution melt (HRM) PCR, hot start PCR, in-situ PCR, intersequence specific (ISS) PCR, inverse PCR, linear-after-the-exponential PCR (LATE PCR), ligation-mediated PCR, long-range PCR, methylation-specific (MSP) PCR, miniprimer PCR, multiplex PCR, nanoparticle-assisted PCR (nanoPCR), nested PCR, overlap extension PCR (OE-PCR), quantitative PCR (qPCR) (also called real-time PCR), reverse-transcriptase real-time PCR (RT-qPCR), RNase H-dependent PCR, single specific primer PCR (SSP-PCR), solid phase PCR (SP-PCR), suicide PCR, thermal asymmetric interlaced PCR (TAIL-PCR), touch down PCR, variable number tandem repeat and (VNTR) PCR. In some embodiments, the PCR is a qPCR. In embodiments, as described herein, the biological sample is analyzed using qPCR.

A biological sample can include tissue, blood, hair, saliva, cells (e.g., cell culture, cell biopsy, buccal cells, etc.), urine, feces, extracted DNA, and extracted RNA, a dried blood spot on a sample carrier, a dried blood spot on paper, a dried blood spot on a dried blood spot card, a dried blood spot on filter paper, a saliva sample on a sample carrier, a saliva sample on paper, a saliva sample on a saliva sample card, a saliva sample on filter paper among other biological sample types. A biological sample can be collected on a swab. For example, a swab can collect a biological sample from a throat (e.g., cells and/or mucosa), a cheek swab (e.g., cells, buccal cells, and/or mucosa), nasal tissue (e.g., cells, and/or mucosa), and combinations thereof. A swab can collect a biological sample from a surface. For example, a swab can be used to collect a biological sample from a floor (e.g., ground), a table, a wall, a ceiling, or an object. A blood sample can be understood to include and/or comprise liquid blood such as whole blood, plasma, or serum, or dried blood. A liquid blood sample can be obtained from a subject through a variety of methods such as, capillary sampling, arterial sampling, and venipuncture. A dried blood sample may be obtained on any number of sample carriers such as, but not limited to, a dried blood spot card, a filter paper, a swab, and/or a tube. In some embodiments, the biological sample is a biological sample blotted and dried on or in a sample carrier. In some embodiments, the sample carrier comprises an absorbent surface including, but not limited to, an absorbent paper, a filter paper, a glass fiber strip, and/or a glass fiber membrane. In some embodiments, the sample carrier comprises a cartridge or a housing. In some embodiments, the sample carrier comprises a desiccant. In some embodiments, the sample carrier comprises indicia indicating a sample area configured to receive the biological sample.

A biological sample can be collected on a swab. For example, a swab can collect a biological sample from a throat (e.g., cells and/or mucosa), a cheek swab (e.g., cells, buccal cells, and/or mucosa), nasal tissue (e.g., cells, and/or mucosa), and combinations thereof. A swab can collect a biological sample from a surface. For example, a swab can be used to collect a biological sample from a floor (e.g., ground), a table, a wall, a ceiling, or an object.

The volume of the blood sample tested may be about 5 μl to about 10 about 25 μl to about 50 μl, about 100 μl to about 1000 μl (e.g., about 50 μl to about 100 μl, about 100 μl to about 200 μl, about 200 μl to about 800 μl, about 200 μl to about 600, about 200 μl to about 400 μl). Accordingly, the volume of the blood sample tested may thus be about 10 μl, about 20 μl, about 30 μl, about 40 μl, about 50 μl, 60 μl, about 70 μl, about 80 μl, about 90 μl, or about 100 about 150 or about 200 μl.

When the biological sample includes saliva, the saliva can be collected from a subject using any method suitable for saliva collection. The saliva sample may be or comprise a dried saliva sample or a liquid saliva sample.

In some embodiments, the volume of the saliva tested may be about 0.5 mL to about 3.0 mL (e.g., about 0.5 mL to about 1 mL, about 1 mL to about 1.5 mL, about 1.5 mL to about 2.0 mL, about 2.0 mL to about 2.5 mL, about 2.5 mL to about 3 mL) to about 1000 μl, about 1000 μl to about 1500 μl, about 1500 μl to about 800 μl, about 200 μl to about 600, about 200 μl to about 400 μl). The volume of the saliva sample tested may thus be about 0.5 mL, about 1 mL, about 1.5 mL, about 2.0 mL, about 2.5 mL, or about 3.0 mL. In embodiments, the volume of saliva may be about 1 ml to about 3 ml.

In embodiments where the biological sample is and/or comprises a tissue and/or a cell sample, the tissue and/or cell sample may be and/or comprise a buccal sample. A buccal sample may comprise a dried buccal sample. A buccal sample may be collected from a subject by scraping the surface of a portion of skin of a subject, including, for example, scraping the cheek of a subject using a swab.

The biological sample can be positioned on a sample carrier. For example, the sample carrier can be paper, and the biological sample may be allowed to dry, such as in e.g., a dried blood spot (DBS). For example, a DBS can be created when liquid blood is placed on paper and allowed to dry. In some embodiments, the biological sample is a dried saliva spot (DSS). For example, a DSS can be created when liquid saliva is placed on paper and allowed to dry. When the biological sample is a tissue and/or cell (e.g., buccal sample), the tissue and/or cell (e.g., buccal) sample can be placed on paper and allowed to dry.

The paper can be absorbent and/or can be chemically treated, where such chemical treatment can be to facilitate DNA extraction and/or DNA elution. Non limiting examples of paper include, PERKIN ELMER 266 FIVE SPOT RUO CARD®, WHATMAN® (absorbent filter paper), WHATMAN® FTA® Cards (chemically treated absorbent paper), FLINDERS TECHNOLOGY ASSOCIATES ° (paper), AVANTEC MFS ° (paper), Ahlstrom grade 226 (A-226) (e.g., filter paper), and Munktell TFN (M-TFN) (e.g., filter paper). In some embodiments, the volume of the biological sample positioned onto the sample carrier (e.g., paper) may be about 5 μl to about 10 μl, about 10 μl to about 25 μl, about 25 μl to about 50 μl, about 50 μl to about 1000 μl (e.g., about 50 μl to about 100 μl, about 100 μl to about 200 μl, about 200 μl to about 1000 μl, about 300 μl to about 1000 μl, about 400 μl to about 1000 μl, about 500 μl to about 1000 μl, about 600 μl to about 1000 μl, about 700 μl to about 1000 μl, about 800 μl to about 1000 μl, or about 900 μl to about 1000 μl). The volume of biological sample positioned on the paper may be about 50 μl about 100 μl, about 200 μl, about 500 μl, or about 1000 μl.

At least a portion of the dried biological sample can be used for testing. In some embodiments, DNA can be eluted from a dried biological sample, such as a portion of a dried biological sample removed from a dried spot on a piece of paper. The biological sample dried on the piece of paper can have a diameter that is about 0.25 mm to about 10 mm in diameter, although the disclosure is not limited to the size of a sample, and such examples are provided for illustration and not limitation. For example, a biological sample can be about 0.25 mm in diameter, about 0.5 mm in diameter, about 1.0 mm in diameter, about 1.5 mm in diameter, about 2.0 mm in diameter, about 2.5 mm in diameter, about 3.0 mm in diameter, about 3.5 mm in diameter, about 4.0 mm in diameter, about 4.5 mm in diameter, about 5.0 mm in diameter, about 5.5 mm in diameter, about 6.0 mm in diameter, about 6.5 mm in diameter, about 7.0 mm in diameter, about 7.5 mm in diameter, about 8.0 mm in diameter, about 8.5 mm in diameter, about 9.0 mm in diameter, or about 10.0 mm in diameter or larger. In some embodiments, the biological sample is about 0.5 mm to about 5.0 mm in diameter, such as, e.g., about 1.5 mm in diameter or about 3.0 mm in diameter. In some embodiments, the biological sample is about 3.2 mm in diameter, or about 3.5 mm in diameter.

DNA can be eluted from a biological sample into a vessel. For example, DNA can be eluted from a blood sample (e.g., a liquid blood sample or a DBS), using an elution solution that may include or comprise one or more PCR reagents. In some embodiments, the elution solution comprises water, and may comprise deionized water. The elution solution may comprise a base and a buffer. For example, the buffer and the base can be combined in a 1:1 ratio in the solution (e.g., the elution solution). In such embodiments, the buffer and the base (e.g., in a 1:1 ratio) can further comprise one or more qPCR reagents as described herein.

The buffer may comprise and/or be selected from the group consisting of Tris buffer, (e.g., Tris base, Tris-HCL, and/or TRIZMAP), and combinations thereof. For example, the buffer may be Tris buffer and the base may be KOH. In some embodiments, the buffer may be TRIZMA® and the base may be KOH. The base may be at a concentration of about 5 mM to about 50 mM, (e.g., about 10 mM to about 50 mM, about 15 mM to about 50 mM, 20 mM to about 50 mM, 25 mM to about 50 mM, 30 mM to about 50 mM, 35 mM to about 50 mM, 40 mM to about 50 mM, 45 mM to about 50 mM). In some embodiments, the base may be at a concentration of about 20 mM.

The buffer may be at a concentration of about 10 mM to about 50 mM, (e.g., about 10 mM to about 50 mM, about 15 mM to about 50 mM, 20 mM to about 50 mM, 25 mM to about 50 mM, 30 mM to about 50 mM, 35 mM to about 50 mM, 40 mM to about 50 mM, 45 mM to about 50 mM.) In some embodiments, the buffer may be at a concentration of about 30 mM.

The biological sample (e.g., a liquid blood sample, a DBS, a liquid saliva sample, a tissue sample, a dried saliva sample (DSS), etc.) and the elution solution may be incubated in a vessel. For example, a blood sample (e.g., a liquid blood sample or a DBS), an elution solution, and a reaction solution comprising one or more qPCR reagents, can be incubated in a vessel. The incubation can comprise incubating at a first temperature to elute the biological sample, and incubating at a second temperature to perform qPCR. The elution solution comprising one or more qPCR reagents may further comprise components (e.g., a buffer and a base as described above) to facilitate the elution of DNA from the biological sample during the incubation. The incubation may be about 5 minutes to about 60 minutes, (e.g., about 5 minutes to about 60 minutes, about 10 minutes to about 60 minutes, about 15 minutes to about 60 minutes, about 20 minutes to about 60 minutes, about 25 minutes to about 60 minutes, about 30 minutes to about 60 minutes, about 35 minutes to about 60 minutes, about 40 minutes to about 60 minutes, about 45 minutes to about 60 minutes, about 50 minutes to about 60 minutes, about 55 minutes to about 60 minutes). In some embodiments, the incubation may be about 30 minutes.

In another example, biological sample (e.g., a liquid blood sample, a DBS, a liquid saliva sample, a tissue sample, a dried saliva sample (DSS), etc.) and the elution solution may be incubated separately from a reaction solution. In other words, in some embodiments, the biological sample and the elution solution do not contact the reaction solution during incubation. In some embodiments, the elution solution, comprising a buffer and a base (as described above), facilitates the elution of DNA from the biological sample during the incubation. In some embodiments, the incubation of the elution solution and the biological sample may be about 5 minutes to about 60 minutes, (e.g., about 5 minutes to about 60 minutes, about 10 minutes to about 60 minutes, about 15 minutes to about 60 minutes, about 20 minutes to about 60 minutes, about 25 minutes to about 60 minutes, about 30 minutes to about 60 minutes, about 35 minutes to about 60 minutes, about 40 minutes to about 60 minutes, about 45 minutes to about 60 minutes, about 50 minutes to about 60 minutes, about 55 minutes to about 60 minutes). In some embodiments, the incubation of the elution solution and the biological sample may be about 20 minutes.

In some embodiments, the biological sample is contacted with about 50 μl to about 200 μl of the elution solution (e.g., about 50 μl to about 60 about 60 μl to about 70 about 70 μl to about 80 about 80 μl to about 90 about 90 μl to about 100 about 100 μl to about 110 about 110 μl to about 120 μl, about 120 μl to about 130 about 130 μl to about 140 about 140 μl to about 150 about 150 μl to about 160 about 160 μl to about 170 μl, about 170 μl to about 180 about 180 μl to about 190 or about 190 μl to about 200 μl). In some embodiments, the biological sample is contacted with about 80 μl to about 160 μl of the elution solution.

In another example, the vessel comprising the biological sample (e.g., a liquid blood sample, a DBS, a liquid saliva sample, a tissue sample, a DSS, etc.), the elution solution, and a reaction solution comprising one or more PCR reagents (e.g., one or more qPCR reagents) may be incubated in the vessel at a first temperature of at least about 15° C. to about 100° C., (e.g., about 15° C. to about 100° C., 20° C. to about 100° C., 25° C. to about 100° C., about 30° C. to about 100° C., about 35° C. to about 100° C., about 40° C. to about 100° C., about 45° C. to about 100° C., about 50° C. to about 100° C., about 55° C. to about 100° C., about 60° C. to about 100° C., about 65° C. to about 100° C., about 70° C. to about 100° C., about 75° C. to about 100° C., about 80° C. to about 100° C., about 85° C. to about 100° C., about 90° C. to about 100° C., or about 95° C. to about 100° C.). In some embodiments, the biological sample, the elution solution, and a reaction solution comprising one or more PCR reagents (e.g., one or more qPCR reagents) are incubated in the vessel at a temperature of about 95° C.

In some embodiments, the biological sample (e.g., a liquid blood sample, a DBS, a liquid saliva sample, a tissue sample, a DSS, etc.) and the elution solution may be incubated in at a temperature of at least about 15° C. to about 100° C., (e.g., about 15° C. to about 75° C., 20° C. to about 75° C., 25° C. to about 75° C., about 30° C. to about 75° C., about 35° C. to about 75° C., about 40° C. to about 75° C., about 45° C. to about 75° C., about 50° C. to about 75° C., about 55° C. to about 75° C., about 60° C. to about 75° C., about 65° C. to about 75° C., about 66° C. to about 74° C., about 67° C. to about 73° C., about 68° C. to about 72° C., about 69° C. to about 71° C., about 70° C. to about 72° C., about 70° C. to about 73° C., about 70° C. to about 74° C., about 70° C. to about 75° C., about 65° C. to about 76° C., about 65° C. to about 77° C., about 65° C. to about 78° C., about 65° C. to about 79° C., about 65° C. to about 80° C., about 65° C. to about 85° C., about 65° C. to about 90° C., about 65° C. to about 95° C., or about 65° C. to about 100° C.). In some embodiments, the biological sample and the elution solution are incubated in the vessel at a temperature of about 70° C.

In some embodiments, the biological sample (e.g., a liquid blood sample, a DBS, a liquid saliva sample, a tissue sample, a dry saliva sample (DSS), a sample carrier comprising a DBS, etc.) and the elution solution may be subjected to a shaking motion (e.g., using an orbital shaker or an orbital shaker platform) during incubation at a speed of about at least 50 revolutions per minute (RPM) to about 2000 RPM (e.g., about 50 RPM to about 800 RPM, about 100 RPM to about 800 RPM, about 200 RPM to about 800 RPM, about 300 RPM to about 800 RPM, about 400 RPM to about 800 RPM, about 500 RPM to about 800 RPM, about 600 RPM to about 700 RPM, about 600 RPM to about 750 RPM, about 600 RPM to about 800 RPM, about 650 RPM to about 700 RPM, about 650 RPM to about 725 RPM, about 650 RPM to about 750 RPM, about 650 RPM to about 775 RPM, about 650 RPM to about 800 RPM, about 700 RPM to about 725 RPM, about 700 RPM to about 750 RPM, about 700 RPM to about 775 RPM, about 700 RPM to about 800 RPM, about 650 RPM to about 900 RPM, about 650 RPM to about 1000 RPM, about 650 RPM to about 1100 RPM, about 650 RPM to about 1200 RPM, about 650 RPM to about 1300 RPM, about 650 RPM to about 1400 RPM, or about 650 RPM to about 1500 RPM). In some embodiments, the biological sample (e.g., a liquid blood sample, a DBS, a liquid saliva sample, a tissue sample, a dry saliva sample (DSS), a sample carrier comprising a DBS, etc.) and the elution solution may be subjected to a shaking motion during incubation at a speed of about 700 RPM.

The elution of the DNA and the qPCR may be performed in situ and may be performed without a pre-amplification step. For example, combination of the biological sample on a sample carrier, the elution solution, and the reaction solution comprising one or more qPCR reagents can be incubated in the vessel to elute DNA from the biological sample, where the eluted DNA is used as a template for the qPCR in situ. Stated differently, the biological sample (e.g., the DBS) is incubated with the sample carrier, the elution solution, and the reaction solution comprising the qPCR reagents, and the eluted DNA is used directly (e.g., without a washing or pre-amplification step) in a qPCR reaction in the same vessel. In such an embodiment, the qPCR reaction can be performed immediately (e.g., within 5 minutes) after the incubation. For example, the DNA used for qPCR can be eluted from the biological sample into the elution solution and the reaction solution comprising one or more qPCR reagents, where the elution and qPCR are performed within the same vessel without pre-amplification of the DNA or a washing of the elutant and/or biological sample and within 5 minutes after the incubation.

In some examples, the elution of the DNA may be performed separately from qPCR and may be performed without a pre-amplification step. For example, in some embodiments, after the incubation of the elution solution and the biological sample is performed (e.g., after DNA is eluted from the biological sample), a vessel comprising one or more reaction reagents dried thereon is contacted with the eluted DNA. In some embodiments, the reaction solution (comprising the one or more reaction reagents) is dried on a surface of the vessel (e.g., on a bottom surface of a well of the vessel). In some embodiments, the reaction solution is air-dried on a surface of the vessel. In some embodiments, the reaction solution is dried on a surface of the vessel at room temperature (e.g., about 20° C. to about 25° C.). In some embodiments, the reaction solution is dried on a surface of the vessel at a temperature ranging from at least about 40° C. to about 100° C. (e.g., about 40° C. to about 85° C., about 45° C. to about 85° C., about 50° C. to about 85° C., about 55° C. to about 85° C., about 60° C. to about 85° C., about 65° C. to about 85° C., about 70° C. to about 85° C., about 75° C. to about 85° C., about 75° C. to about 80° C., about 76° C. to about 81° C., about 77° C. to about 82° C., about 78° C. to about 83° C., about 79° C. to about 84° C., about 80° C. to about 82° C., about 80° C. to about 83° C., about 80° C. to about 84° C., about 80° C. to about 85° C., about 75° C. to about 80° C., about 75° C. to about 90° C., about 75° C. to about 95° C., or about 75° C. to about 100° C.). In some embodiments, the reaction solution is dried on a surface of the vessel at a temperature of about 80° C.

In some embodiments, the reaction solution is dried on a surface of the vessel for at least about 15 minutes (min.) to about 60 min. (e.g., for about 15 min. to about 45 min., about 20 min. to about 45 min., about 25 min. to about 45 min., about 26 min. to about 40 min., about 27 min. to about 40 min., about 28 min. to about 40 min., about 29 min. to about 40 min., about 30 min. to about 40 min., about 31 min. to about 40 min., about 32 min. to about 40 min., about 33 min. to about 40 min., about 34 min. to about 40 min., about 35 min. to about 40 min., about 36 min. to about 40 min., about 37 min. to about 40 min., about 38 min. to about 40 min., about 39 min. to about 40 min., about 26 min. to about 45 min., about 27 min. to about 45 min., about 28 min. to about 45 min., about 29 min. to about 45 min., about 30 min. to about 45 min., about 31 min. to about 45 min., about 32 min. to about 45 min., about 33 min. to about 45 min., about 34 min. to about 45 min., about 35 min. to about 45 min., about 36 min. to about 45 min., about 37 min. to about 45 min., about 38 min. to about 45 min., about 39 min. to about 45 min., about 40 min. to about 45 min., about 25 min. to about 50 min., about 25 min. to about 55 min., about 25 min. to about 60 min. about 35 min. to about 50 min., about 35 min. to about 55 min., about 35 min. to about 60 min. about 35 min. to about 50 min., about 35 min. to about 55 min., or about 35 min. to about 60 min.). In some embodiments, the reaction solution is dried on a surface of the vessel for about 30 to about 40 min. In some embodiments, the reaction solution is deposited on a surface of the vessel and then stored in an oven (e.g., a forced air oven or a drying oven) at any one of the temperatures previously disclosed for any one of the time periods previously disclosed to air-dry the reaction solution on the surface of the vessel.

In some embodiments, at least about 5 μl to about 50 μl (e.g., about 5 μl to about 20 about 5 μl to about 25 about 5 μl to about 30 about 5 μl to about 35 about 5 μl to about 40 about 5 μl to about 45 about 5 μl to about 50 about 10 μl to about 20 about 10 μl to about 25 about 10 μl to about 30 about 10 μl to about 35 about 10 μl to about 40 about 10 μl to about 45 about 10 μl to about 50 about 11 μl to about 25 about 12 μl to about 25 about 13 μl to about 25 about 14 μl to about 25 about 15 μl to about 25 about 16 μl to about 25 about 17 μl to about 25 about 18 μl to about 25 about 19 μl to about 25 about 15 μl to about 20 about 16 μl to about 20 about 17 μl to about 20 about 18 μl to about 20 about 15 μl to about 23 about 15 μl to about 25 about 15 μl to about 30 about 15 μl to about 35 about 15 μl to about 40 about 15 μl to about 45 or about 15 μl to about 50 μl) of the eluted DNA is contacted with the vessel comprising the air-dried reaction reagents. In some embodiments, about 15 μl to about 20 μl of the eluted DNA is contacted with the vessel comprising the air-dried reaction reagents. In some embodiments, the eluted DNA is used to re-suspend the dried reaction reagents. In some embodiments, qPCR can be performed after the reaction reagents are re-suspended with the eluted DNA.

In some embodiments, setting up the biological sample for qPCR (e.g., re-suspending the dried reaction reagents with the eluted DNA, sealing the vessel, etc.) can be done in at least about 3 min. to about 10 min. (e.g., about 3 min. to about 4 min., about 3 min. to about 4 min., about 3 min. to about 5 min., about 3 min. to about 6 min., about 3 min. to about 7 min., about 3 min. to about 8 min., about 3 min. to about 9 min., about 3 min. to about 10 min., about 4 min. to about 5 min., about 4 min. to about 6 min., about 4 min. to about 7 min., about 4 min. to about 8 min., about 4 min. to about 9 min., about 4 min. to about 10 min., about 5 min. to about 6 min., about 5 min. to about 7 min., about 5 min. to about 8 min., about 5 min. to about 9 min., or about 5 min. to about 10 min.). In some embodiments, setting up the biological sample for qPCR (e.g., re-suspending the dried reaction reagents with the eluted DNA, sealing the vessel, etc.) can be done in about 5 min.

In some embodiments, performing qPCR on the biological sample can be done in at least about 55 min. to about 75 min. (e.g., about 55 min. to about 60 min., about 55 min. to about 65 min., about 55 min. to about 70 min., about 55 min. to about 75 min., about 58 min. to about 59 min., about 61 min. to about 65 min., about 62 min. to about 65 min., about 63 min. to about 65 min., about 54 min. to about 65 min., about 60 min. to about 65 min., about 60 min. to about 70 min., or about 60 min. to about 75 min.). In some embodiments, performing qPCR on the biological sample can be done in about 60 min.

In some embodiments, analyzing the qPCR results can be done in at least about 5 min. to about 25 min. (e.g., about 5 min. to about 10 min., about 5 min. to about 15 min., about 5 min. to about 20 min., or about 5 min. to about 25 min., about 10 min. to about 15 min., about 10 min. to about 20 min., or about 10 min. to about 25 min.). In some embodiments, analyzing the qPCR results can be done in about 10 min.

In some embodiments, qPCR can utilize fluorescent probes for detection. A blood sample such as a DBS or a liquid blood sample can introduce heme protein into the qPCR reaction. Heme protein can be an inhibitor to qPCR and can have a broad florescent spectrum which can cause interference in the detection of a signal coming from qPCR probes used in an in situ qPCR reaction. Performing qPCR on DNA eluted from a DBS sample can be difficult due to inhibitors. For example, eluting DNA from a DBS sample can introduce inhibitors such as heme protein when the DNA is eluted in situ. The inhibitors can be overcome by the use of a polymerase and/or master mixes configured for qPCR.

Non-limiting examples of polymerase are PERFECTA® MULTIPLEX QPCR TOUGHMIX® (Taq DNA Polymerase), SENSIFAST® (uses hot-start Taq DNA polymerase), LUNA UNIVERSAL QPCR MASTER MIX® (uses hot-start Taq DNA polymerase), Platinum II HOT-START PCR MASTER MIX® (hot-start DNA Polymerase), TAQPATH PROAMP MASTER MIX® (Taq polymerase), AB SCRIPT II ONE STEP RT-QPCR PROBE KIT® (hot-start Taq), and FORGET-ME-NOT™ qPCR Master Mix (Taq polymerase). Inhibition resistant polymerases can be used. For example, TAQPATH PROAMO MASTER MIX® and OMNITAQ®. Accordingly, the one or more qPCR reagents can include enzymes resistant to inhibitors (e.g., TAQPATH PROAMP MASTER MIX® (Taq polymerase)). The one or more qPCR reagents can comprise deoxyuridine triphosphate (dUTP).

As mentioned herein, the incubating of the combination of the biological sample on a sample carrier, the elution solution, and the reaction solution comprising one or more qPCR reagents comprises incubating the combination at a first temperature to elute the biological sample, and incubating at least at a second temperature to perform qPCR. In some examples, the biological sample is incubated with the elution solution at a first temperature, and the eluted DNA is used to resuspend the air-dried reaction reagents prior to performing qPCR at a second temperature. For example, a denaturation step of the qPCR reaction can be performed at the second temperature. The qPCR reaction may include a denaturation step that can be performed at a temperature of about 50° C. to about 100° C. (e.g., about 55° C. to about 100° C., about 60° C. to about 100° C., about 65° C. to about 100° C., about 70° C. to about 100° C., about 75° C. to about 100° C., about 80° C. to about 100° C., about 85° C. to about 100° C., about 90° C. to about 100° C., or about 95° C. to about 100° C.), for about 5 seconds to about 60 seconds, (e.g., about 5 seconds to about 60 seconds, about 10 seconds to about 60 seconds, about 15 seconds to about 60 seconds, about 20 seconds to about 60 seconds, about 25 seconds to about 60 seconds, about 30 seconds to about 60 seconds, about 35 seconds to about 60 seconds, about 40 seconds to about 60 seconds, about 45 seconds to about 60 seconds, about 50 seconds to about 60 seconds, about 55 seconds to about 60 seconds). In some embodiments, the first temperature is about the same as the second temperature. In some embodiments, the first temperature is different than the second temperature.

An annealing/extension may be performed at a temperature of about 50° C. to about 100° C., (e.g., about 55° C. to about 100° C., about 60° C. to about 100° C., about 65° C. to about 100° C., about 70° C. to about 100° C., about 75° C. to about 100° C., about 80° C. to about 100° C., about 85° C. to about 100° C., about 90° C. to about 100° C., or about 95° C. to about 100° C.), for about 5 seconds to about 60 seconds, (e.g., about 5 seconds to about 60 seconds, about 10 seconds to about 60 seconds, about 15 seconds to about 60 seconds, about 20 seconds to about 60 seconds, about 25 seconds to about 60 seconds, about 30 seconds to about 60 seconds, about 35 seconds to about 60 seconds, about 40 seconds to about 60 seconds, about 45 seconds to about 60 seconds, about 50 seconds to about 60 seconds, about 55 seconds to about 60 seconds).

In some embodiments, the denaturation and annealing/extension can be repeated for one or more cycles. The one or more cycles may be about 10 to about 50 cycles, (e.g., 10 to about 50 cycles, about 12 to about 50 cycles, about 14 to about 50 cycles, about 16 to about 50 cycles, about 18 to about 50 cycles, about 20 to about 50 cycles, about 22 to about 50 cycles, about 24 to about 50 cycles, about 24 to about 50 cycles, about 26 to about 50 cycles, about 28 to about 50 cycles, about 30 to about 50 cycles, about 32 to about 50 cycles, about 34 to about 50 cycles, about 36 to about 50 cycles, about 38 to about 50 cycles, about 40 to about 50 cycles, about 42 to about 50 cycles, about 44 to about 50 cycles, about 46 to about 50 cycles, or about 48 to about 50 cycles).

In addition to a polymerase, a qPCR reaction can include one or more additional reagents. In some embodiments, reaction solution comprising the one or more qPCR reagents may comprise one or more deoxynucleotide triphosphates (dNTPs), including adenine triphosphate (dATP), cytosine triphosphate (dCTP), guanine triphosphate (dGTP), and thymine triphosphate (dTTP). The one or more qPCR reagents comprises one or more salts. In some embodiments, the salt is selected from the group consisting of ammonium salts (e.g., NH₄Cl), potassium salts (e.g., KCl), magnesium salts (e.g., MgCl₂), and combinations thereof. The one or more qPCR reagents may be adjusted for the qPCR reaction as is understood in the art. In some embodiments, the one or more qPCR reagents can be at the desired concentration in the solution within the vessel.

The one or more salts may be at a concentration of about 0.5 mM to about 5.0 mM. The one or more salts may be, for example, a magnesium salt at a concentration of about 0.5 mM to about 5.0 mM. For example, the magnesium salt can be in the solution at a concentration of about 0.5 mM, about 1.0 mM, about 1.5 mM, about 2.0 mM, about 2.5 mM, about 3.0 mM, about 3.5 mM, about 4.0 mM, about 4.5 mM, or about 5.0 mM. In some embodiments, the one or more salts is a potassium salt at a concentration of about 35 mM to about 100 mM. For example, potassium salt can be in the solution at a concentration of about 35 mM, about 40 mM, about 45 mM, about 50 mM, about 45 mM, about 50 mM, about 55 mM, about 60 mM, about 65 mM, about 70 mM, about 75 mM, about 80 mM, about 85 mM, about 90 mM, about 95 mM, or about 100 mM.

In some embodiments, the one or more qPCR reagents include dimethylsulfoxide (DMSO) at a concentration of about 1% to about 10% v/v. For example, DMSO can be in the solution at a concentration of about 1%, about 2.0% v/v, about 3.0% v/v, about 4.0% v/v, about 5.0% v/v, about 6.0% v/v, about 7.0% v/v, about 8.0% v/v, about 9.0% v/v, or about 10.0% v/v. The one or more qPCR reagents may comprise formamide at a concentration of about 1% to about 10% v/v. For example, formamide can be in the solution at a concentration of about 1% v/v, about 2.0% v/v, about 3.0% v/v, about 4.0% v/v, about 5.0% v/v, about 6.0% v/v, about 7.0% v/v, about 8.0% v/v, about 9.0% v/v, or about 10.0% v/v.

The one or more qPCR reagents may comprise bovine serum albumin (BSA) at a concentration of about 10 μg/ml to about 100 μg/ml. For example, BSA can be present in the solution at a concentration of about 10 μg/ml, about 15 μg/ml, about 20 μg/ml, about 25 μg/ml, about 30 μg/ml, about 35 μg/ml, about 40 μg/ml, about 45 μg/ml, about 50 μg/ml, about 55 μg/ml, about 60 μg/ml, about 65 μg/ml, about 70 μg/ml, about 75 μg/ml, about 80 μg/ml, about 85 μg/ml, about 90 μg/ml, about 95 μg/ml, or about 100 μg/ml.

The one or more qPCR reagents may comprise betaine at a concentration of about 0.5 M to about 2.5 M. For example, betaine can be present in the solution at a concentration of about 0.5 M, about 1.0 M, 1.5 M, about 2.0 M, or about 2.5 M.

The one or more qPCR reagents may include an antibody. For example, an antibody can be included as a qPCR reagent during a hot-start qPCR reaction, where the antibody can block the polymerase enzyme until it is heat inactivated.

The one or more qPCR reagents may comprise one or more probes, DNA binding dyes and/or one or more primers. For example, the one or more qPCR reagents may be a fluorescent probe and/or one or more fluorescent primers. The one or more qPCR reagents may comprise a preservative, such as, e.g., a biocide (e.g., ProClin950®).

The one or more qPCR agents may include one or more stabilizers that may comprise and/or be selected from a group consisting of trehalose, mannitol, BSA, polyethylene, and combinations thereof. The stabilizer may be lyophilized. The one or more qPCR reagents can include an indicator that can comprise and/or be selected from the group consisting of cresol red, bromophenol blue, and xylene cyanol, with such examples provided for illustration only. In some embodiments, the indicator is cresol red. The one or more qPCR reagents may comprise water, such as nuclease-free water and/or deionized water.

The one or more qPCR reagents may be lyophilized in the vessel. For example, one or more of the qPCR reagents mentioned herein can be added to the vessel and lyophilized or lyophilized prior to being added to the vessel. The vessel comprising the qPCR reagents may be stored for a period of time of about 1 day to about 1 year. For example, the vessel comprising the one or more qPCR reagents can be stored for a period of time of about 1 day, about 2 days, about 3, days, about 4 days, about 5 days, about 6 days, about 7 days, about 8 days, about 9 days, about 10 days, about 2 weeks, about 3, weeks, about 4 weeks, about 1 month, about 2 months, about 3 months, about 4 months, about 5 months, about 6 months, about 7 months, about 8 months, about 9 months, about 10 months, about 11 months, or about 12 months. The qPCR reagents can be reconstituted by the addition of a liquid, such as, e.g., water, deionized water, and/or nuclease free water. The liquid can be a solution to elute DNA from the biological sample. As such, the biological sample, (e.g., a DBS, a DSS, liquid blood sample, a saliva sample, a buccal cell sample, etc.) can be added to the vessel with the lyophilized PCR reagents (e.g., qPCR reagents), and a solution can be added to the vessel to elute DNA from the blood sample and reconstitute the qPCR reagents. In embodiments, the vessel comprising the one or more lyophilized qPCR reagents can be stored, e.g., at a temperature of about −20° C. to about 35° C., (e.g., about −15° C. to about 35° C., about −10° C. to about 35° C., about −5° C. to about 35° C., about 0° C. to about 35° C., about 5° C. to about 35° C., about 10° C. to about 35° C., about 15° C. to about 35° C., about 20° C. to about 35° C., about 25° C. to about 35° C., or about 30° C. to about 35° C.).

Disclosed herein are kits for use in analyzing a biological sample dried on a sample carrier, where in one embodiment, the kits comprise, an elution solution, a reaction solution; and instructions for performing qPCR on a sample carrier having a biological sample dried thereon, the instructions including: (i) creating a combination, the combination comprising the sample carrier with the biological sample dried thereon, the reaction solution, and the elution solution, wherein sample carrier has not been subjected to incubation prior to creating the combination; and (ii) incubating the combination to elute the biological sample and to perform qPCR on the at least a portion of the biological sample, wherein said instructions do not include instructions for subjecting the sample carrier with the biological sample dried thereon to incubation prior to creating the combination, and/or include instructions to avoid subjecting the combination to incubation prior to creating the combination. In an embodiment, the instructions include instructions to create the combination in a vessel. The vessel may comprise a microtiter plate.

### II. Examples

### Example 1. In Situ Four-Analyte Multiplex qPCR Assay with Dried Blood Spot Disc

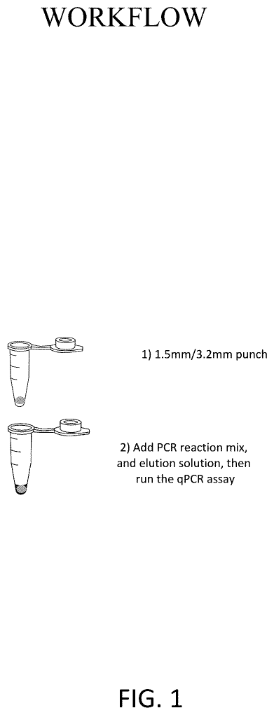

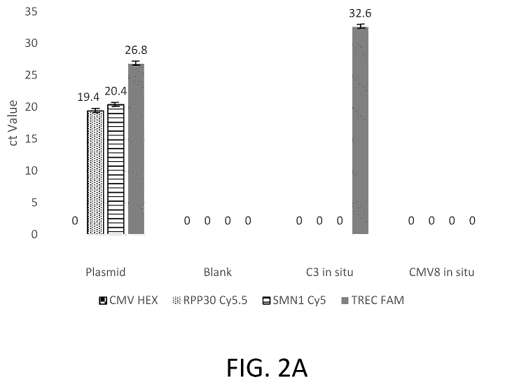

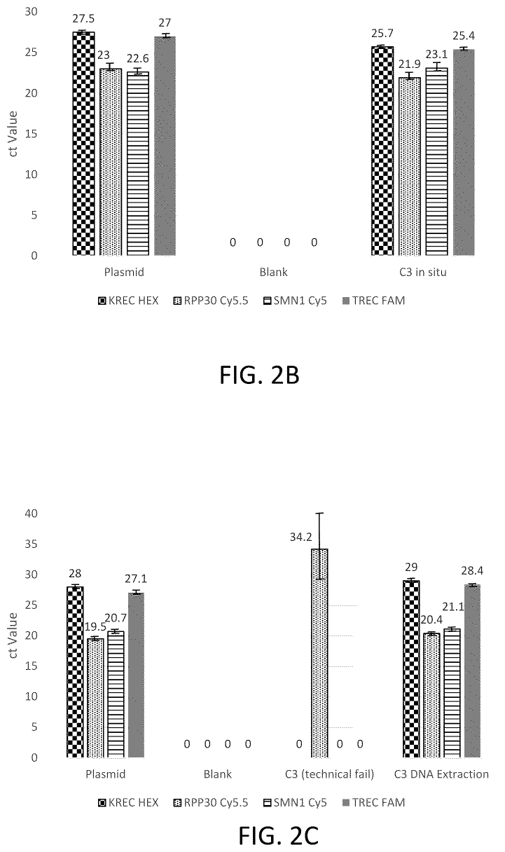

An Exemplary Workflow is Provided in FIG. 1. In Some Samples, from a Dried Blood spot on filter paper, a 1.5 mm disc was removed and placed in a sample well or sample tube. In other samples, whole adult blood spiked with Cytomegalovirus (CMV) was used in place of dried blood spots. A qPCR reaction mixture and a solution of 30 mM Tris and 20 mM KOH were added in a 1:1 ratio to the sample well or sample tube with the DBS-punched disc or whole blood and incubated at 95° C. for 30 min to extract DNA separately from the PCR program. The PCR reaction mixture contained the components listed in Table 1 along with a conventional polymerase and included NEOMDX™ PCR master mix (FIG. 2A) or with some components replaced with those from TAQPATH PROAMP™ master mix (FIG. 2B), as indicated. Analyte targets included TREC, KREC, SMA and RPP plasmids or CMV. As a control, the procedure was also performed on a purified DNA template with a conventional polymerase (C3 DNA extraction or C3 in situ). Plasmid qPCRs had five replicate samples. Blank qPCRs had two replicate samples. The qPCR reactions were performed in 30 μl reactions with the following conditions: 2 min at 37° C., 5 min at 94° C., and 40 cycles of 10 sec at 93° C., 30 sec at 60° C., and 40 sec at 69° C. Ct values (±standard deviation) were calculated and compared between in situ and control qPCRs (see FIGS. 2A-2C). One of five samples tested for C3 in situ amplification of the TREC plasmid was successful when using the NEOMDX™ master mix. Quantitative PCRs with in situ extraction using NEOMDX™ PCR master mix (FIG. 2A and FIG. 2C) or NEOMDX™ PCR master mix with some components replaced with those from TAQPATH PROAMP™ master mix (FIG. 2B) all showed amplification of plasmid DNA similar to amplification of control samples with separate DNA extraction. DNA from all samples was amplified when some components of the NEOMDX™ master mix were replaced with those from TAQPATH PROAMP™ master mix.

TABLE 1
in situ qPCR reaction mix
Multiplex qPCR Components

NeoT Forward (TREC plasmid target)
NeoT Reverse (TREC plasmid target)
NeoT Probe (TREC plasmid target)
NeoS Forward (SMA plasmid target)
NeoS Reverse (SMA plasmid target)
NeoS1 Probe (SMA plasmid target)
NeoS2 Probe (SMA plasmid target)
NeoK Forward (KREC plasmid target)
NeoK Reverse (KREC plasmid target)
NeoK Probe (KREC plasmid target)
NeoRP Forward (RPP plasmid target)
NeoRP Reverse (RPP plasmid target)
NeoRP Probe (RPP plasmid target)
NH₄Cl (g)
KCl solution (μl)
ProClin 950 (μl)
PCR Master Mix
Neo ssDNA (μl)
Cresol Red Sodium Salt (g)
Nuclease-free water
PCR Component stabilizer
Polymerase

### Example 2. In Situ Four-Analyte Multiplex qPCR Assay with Dried Blood Spot Disc and TAQPATH™ Enzyme

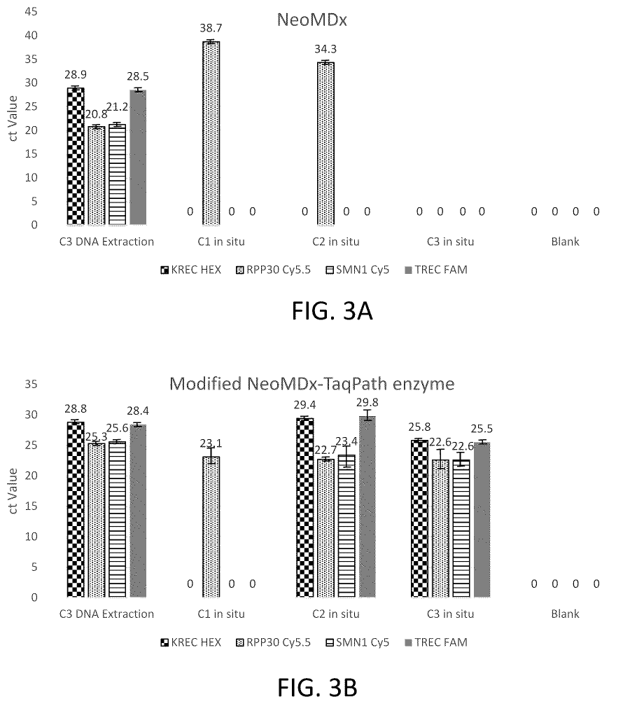

A PCR reaction mixture and a solution of 30 mM Tris and 20 mM KOH were added in a ratio of 1:1 into a sample well or tube with a 1.5 mm dried blood spot punched disc. Discs and reagents were incubated at 95° C. for 30 min before qPCR. The qPCR reactions were performed in 30 μl reactions with the following conditions: 5 min at 94° C., and 40 cycles of 15 sec at 93° C., 30 sec at 60° C., and 40 sec at 69° C. For C3 DNA extraction samples, DNA was extracted and purified using a modified NEOMDX™ master mix in which some components were replaced with components from TAQPATH PROAMP™ master mix. DNA was then mixed with qPCR reagents, and qPCR was performed. Amplification either used NEOMDX™ (FIG. 3A) or TAQPATH™ DNA polymerase enzyme (FIG. 3B) as indicated. Samples included positive controls (C3 DNA extraction), negative controls without DNA template (blank), dried blood spot with only RPP plasmid DNA (C1 in situ), dried blood spot with moderate levels of TREC and KREC and higher levels of RPP and SMN1 plasmid DNA (C2 in situ), or dried blood spot with higher levels of TREC, KREC, RPP and SMN1 (C3 in situ). All analyses were completed in technical triplicate. One in three samples of RPP plasmid were successfully amplified when only RPP plasmid DNA was present (C1 in situ) or when a combination of moderate levels of TREC and KREC plasmid DNA with high levels of RPP and SMN1 plasmid DNAs were present (C2 in situ). Analysis indicated successful amplification of all plasmid DNA with either NEOMDX™ or TAQPATH™ polymerase enzyme on a four-analyte (TREC-KREC-SMN-RPP) multiplexed qPCR analysis with in situ elution of DNA. Amplification of RPP and SMN1 plasmids yield larger ct values with TAQPATH™ polymerase enzyme. DNA from all tested samples was amplified when using NEOMDX™ master mix with TAQPATH™ polymerase enzyme.

### Example 3. In Situ Dry Chemistry qPCR of Four Analytes from Dried Blood Spots

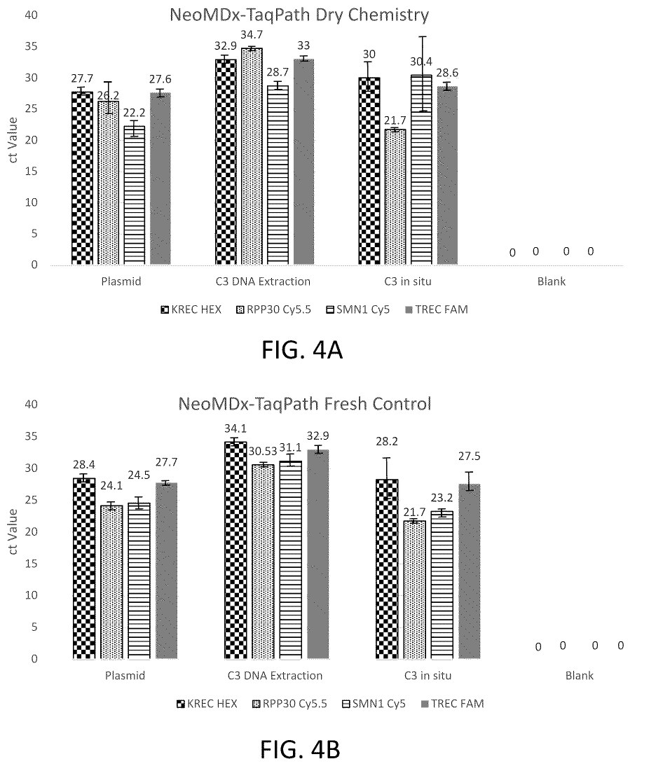

A PCR component stabilizer was added to the qPCR reaction mix (Table 1) and then dried on a qPCR plate using GENEVAC™ evaporators. Dried plates were stored at room temperature for 5 weeks. The dried reaction mixes were resuspended in a sample well with water mixed with a solution of 30 mM Tris and 20 mM KOH and the dried blood spot punch or plasmid DNA mix was added to the well and analyzed directly with qPCR with a TREC-KREC-SMN1-RPP plasmid multiplex assay in triplicate. Fresh qPCR mix was used as a control. Sample incubation/elution was performed during qPCR cycling in a modified 2-step qPCR protocol in 30 μl reaction volumes with the following conditions: initial denaturation and extraction for 30 min at 95° C., and 45 cycles of denaturation for 15 sec at 93° C. and annealing/extension for 60 sec at 60° C. Ct values were analyzed (see FIGS. 4A-4B). One of three tested samples was successfully amplified from RPP plasmid DNA with C3 DNA extraction or C3 in situ DNA extraction. Two of three samples were successfully amplified from KREC or RPP plasmid DNA in C3 in situ DNA extraction. In all cases there was successful amplification of at least one sample from all plasmid PCR targets regardless of whether qPCR reaction mixture was lyophilized and stored or used fresh.

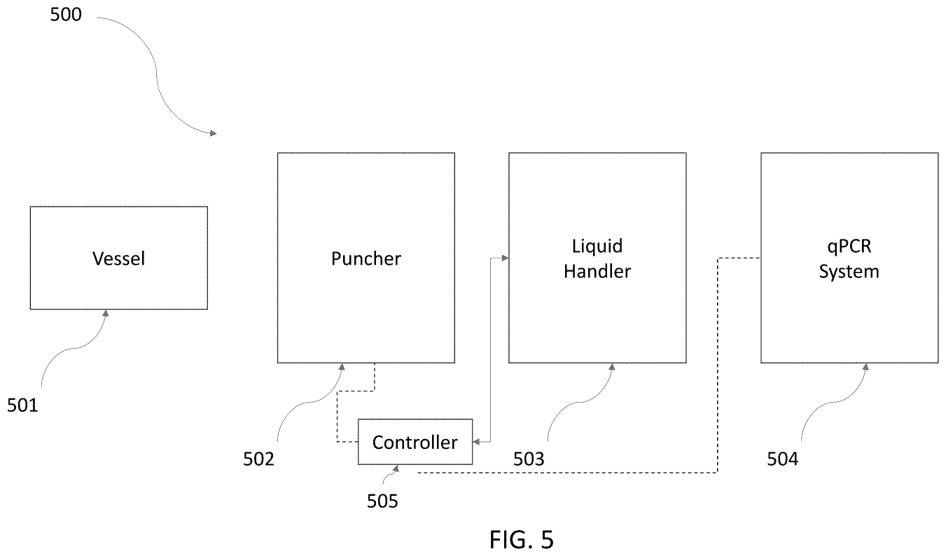

FIG. 5 discloses one embodiment of a system 500 for analyzing a biological sample dried on a sample carrier, wherein the FIG. 5 embodiment of the system comprises a vessel 501, a liquid handler 503, a qPCR system 504 configured to perform qPCR on the biological sample, and, a controller 505 configured to cause the liquid handler to create a combination in the vessel 501, the combination including an elution solution, a reaction solution, and the biological sample dried on the sample carrier, where the sample carrier has not been subjected to incubation prior to creating the combination. As shown in FIG. 5, in some embodiments e.g., where the biological sample is blood, and the sample carrier is a blood spot card, a system 500 may include a puncher 502 for removing a portion of the dried blood spot card that includes the biological sample dried on a portion of the blood spot card, wherein the puncher 502 may be automated or manual. In some embodiments where the vessel 501 is a microtiter plate, the system 500 may include a plate handler (not shown) for moving the vessel/microtiter plate 501 between and/or to-and-from one or more of the puncher 502, liquid handler 503, and/or qPCR system 504.

In some embodiments, the controller 505 that is configured to create the combination is separate from, but in communication, direct or indirect, wired or wireless, with the liquid handler 503. The liquid handler 503 may have its own controller separate from and/or in communication with the controller 505, while in some embodiments, the controller 505 may be integrated with the liquid handler 503. In embodiments, one or more of the (optional) puncher 502, (optional) plate handler, liquid handler 503, and/or qPCR system 504 may have its own controller that may be in communication, direct or indirect, with the controller 505. In some embodiments where the sample carrier is a portion (e.g., dried blood spot) of a dried blood spot card, controller 505 may cause for the processing of the biological sample on the sample carrier by causing a plate handler to provide a vessel 501 to the puncher 502 to allow for the removal of at least a portion of the dried blood spot card (e.g., a portion containing the biological sample) before providing the vessel 501 (e.g., microtiter plate) containing the biological sample dried on the sample carrier to the liquid handler 503 where the controller 505 causes the combination of an elution solution, reaction solution, and the biological sample dried on the sample carrier, to be created in the vessel 501. The vessel 501 may thereafter be provided to the qPCR system 504, for example, by the controller 505, manually, or by some other controller (e.g., plate handling system). In some embodiments, the controller 505 may be part of an optional plate handler system.

As provided herein, all, none, or portions of the workflow of the system 500 may be manual, while portions or significantly most may be automated. Accordingly, those of ordinary skill will thus understand that the controller 505 can be of various forms, and may be part of one or more of the depicted system components (e.g., 502, 503, 504), or separate therefrom, but in either case, optionally in direct or indirect communication with one or more of the components of the system 500 as shown, and further, as described.

Accordingly, it may be understood that one or more of the components (e.g., 502, 503, 504) of a system 500 according to FIG. 5 may comprise another controller that may communicate with controller 505 and/or be considered a part of controller 505. Such component controllers, and/or the controller 505, may thus comprise a processor, which typically may take the form of a microprocessor and/or a computer and suitable software, hardware, and/or firmware for providing the components of the system 500 to create the combination of elution solution, a reaction solution, and at least a portion of the biological sample (dried on the sample carrier), as disclosed herein, where the sample carrier has not been subjected to incubation prior to creating the combination. As provided herein, the controller 505 can be present as a stand-alone processor, to control and coordinate operation of the system 500 for the various modes of operation using the system 500. For this purpose, the controller 500 can be electrically coupled to each of the components (e.g., 502, 503, 504) of the system.

In certain configurations, the controller 505 may be present in one or more computer systems and/or common hardware circuitry including, for example, a microprocessor and/or suitable software for operating the system 500, e.g., one or more of the plate handler, puncher, liquid handler, and/or qPCR system. In some examples, any one or more components of the system 500 can include its own respective processor, operating system and other features to permit operation of that component. The controller 505 can be integral to the components (e.g., 502, 503, 504) or may be present on one or more accessory boards, printed circuit boards or computers electrically coupled to the components (e.g., 502, 503, 504) of the system. The controller 505 is typically electrically coupled to one or more memory units to receive data from the other components (e.g., 502, 503, 504) of the system 500 and permit adjustment of the various system parameters as needed or desired. The controller 500 and/or other processors may be part of a general-purpose computer such as those based on Unix, Intel PENTIUM-type processor, Motorola PowerPC, Sun UltraSPARC, Hewlett-Packard PA-RISC processors, or any other type of processor. One or more of any type computer system may be used according to various embodiments of the technology. Further, the system may be connected to a single computer or may be distributed among a plurality of computers attached by a communications network. It should be appreciated that other functions, including network communication, can be performed and the technology is not limited to having any particular function or set of functions. Various aspects may be implemented as specialized software executing in a general-purpose computer system. The computer system may include a processor connected to one or more memory devices, such as a disk drive, memory, or other device for storing data. Memory is typically used for storing programs, calibrations and data during operation of the system in the various modes. Components of the computer system may be coupled by an interconnection device, which may include one or more buses (e.g., between components that are integrated within a same machine) and/or a network (e.g., between components that reside on separate discrete machines). The interconnection device provides for communications (e.g., signals, data, instructions) to be exchanged between components (e.g., 502, 503, 504) of the system 500. The controller 505 typically is electrically coupled to a power source which can, for example, be a direct current source, an alternating current source, a battery, a fuel cell or other power sources or combinations of power sources. The power source can be shared by the other components of the system. The system 500 may also include one or more input devices, for example, a keyboard, mouse, trackball, microphone, touch screen, manual switch (e.g., override switch) and one or more output devices, for example, a printing device, display screen, speaker. In addition, the system 500 may contain one or more communication interfaces that connect the computer system to a communication network (in addition or as an alternative to the interconnection device). The system may also include suitable circuitry to convert signals received from the various electrical devices present in the system 500. Such circuitry can be present on a printed circuit board or may be present on a separate board or device that is electrically coupled to the printed circuit board through a suitable interface, e.g., a serial ATA interface, ISA interface, PCI interface or the like or through one or more wireless interfaces, e.g., Bluetooth, Wi-Fi, Near Field Communication or other wireless protocols and/or interfaces.

In certain embodiments, the storage system used in the systems described herein typically includes a computer readable and writeable non-volatile recording medium in which codes can be stored that can be used by a program to be executed by the controller 505 or information stored on or in the medium to be processed by the program. The medium may, for example, be a hard disk, solid state drive or flash memory. Typically, in operation, the controller 505 causes data to be read from the non-volatile recording medium into another memory that allows for faster access to the information by the processor than does the medium. This memory is typically a volatile, random access memory such as a dynamic random access memory (DRAM) or static memory (SRAM). It may be located in the storage system or in the memory system. The controller 505 generally manipulates the data within the integrated circuit memory and then copies the data to the medium after processing is completed. A variety of mechanisms are known for managing data movement between the medium and the integrated circuit memory element and the technology is not limited thereto. The technology is also not limited to a particular memory system or storage system. In certain embodiments, the system may also include specially-programmed, special-purpose hardware, for example, an application-specific integrated circuit (ASIC) or a field programmable gate array (FPGA). Aspects of the technology may be implemented in software, hardware or firmware, or any combination thereof. Further, such methods, acts, systems, system elements and components thereof may be implemented as part of the systems described herein or as an independent component. Although specific systems are described by way of example as one type of system upon which various aspects of the technology may be practiced, it should be appreciated that aspects are not limited to being implemented on the described system 500. Various aspects may be practiced on one or more systems having a different architecture or components. The system may comprise a general-purpose computer system that is programmable using a high-level computer programming language. The systems may be also implemented using specially programmed, special purpose hardware. In the systems, the controller 505 comprises a commercially available processor such as the well-known Pentium class processors available from the Intel Corporation. Many other processors are also commercially available. Such a processor usually executes an operating system which may be, for example, the Windows 95, Windows 98, Windows NT, Windows 2000 (Windows ME), Windows XP, Windows Vista, Windows 7, Windows 8 or Windows 10 operating systems available from the Microsoft Corporation, MAC OS X, e.g., Snow Leopard, Lion, Mountain Lion or other versions available from Apple, the Solaris operating system available from Sun Microsystems, or UNIX or Linux operating systems available from various sources. Many other operating systems may be used, and in certain embodiments a simple set of commands or instructions may function as the operating system.

In certain examples, the controller 505 and operating system may together define a platform for which application programs in high-level programming languages may be written. It should be understood that the technology is not limited to a particular system platform, processor, operating system, or network. Also, it should be apparent to those skilled in the art, given the benefit of this disclosure, that the present technology is not limited to a specific programming language or computer system. Further, it should be appreciated that other appropriate programming languages and other appropriate systems could also be used. In certain examples, the hardware or software can be configured to implement cognitive architecture, neural networks or other suitable implementations. If desired, one or more portions of the computer system may be distributed across one or more computer systems coupled to a communications network. These computer systems also may be general-purpose computer systems. For example, various aspects may be distributed among one or more computer systems configured to provide a service (e.g., servers) to one or more client computers, or to perform an overall task as part of a distributed system. For example, various aspects may be performed on a client-server or multi-tier system that includes components distributed among one or more server systems that perform various functions according to various embodiments. These components may be executable, intermediate (e.g., IL) or interpreted (e.g., Java) code which communicate over a communication network (e.g., the Internet) using a communication protocol (e.g., TCP/IP). It should also be appreciated that the technology is not limited to executing on any particular system or group of systems. Also, it should be appreciated that the technology is not limited to any particular distributed architecture, network, or communication protocol.

Certain configurations may be implemented as programmed or non-programmed elements, or any combination thereof. In some instances, the systems 500 may comprise a remote interface such as those present on a mobile device, tablet, laptop computer or other portable devices which can communicate through a wired or wireless interface and permit operation of the systems 500 remotely as desired.

### Example 4. Inhibitor-Tolerant Dry Chemistry qPCR Assay with a One-Step DNA Extraction from Dried Blood Spots

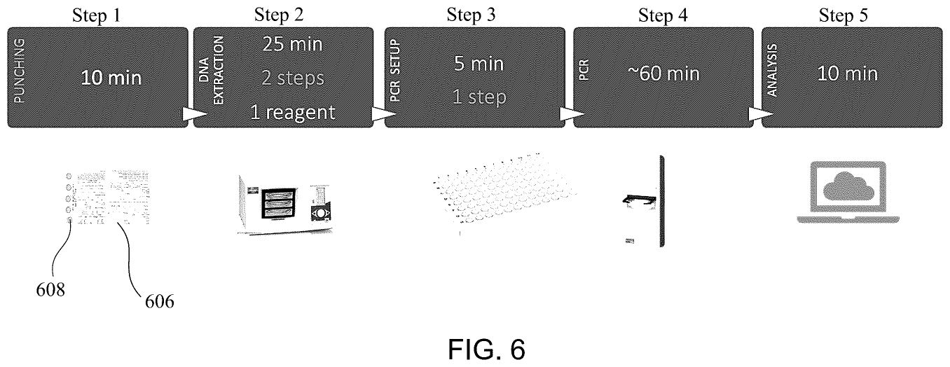

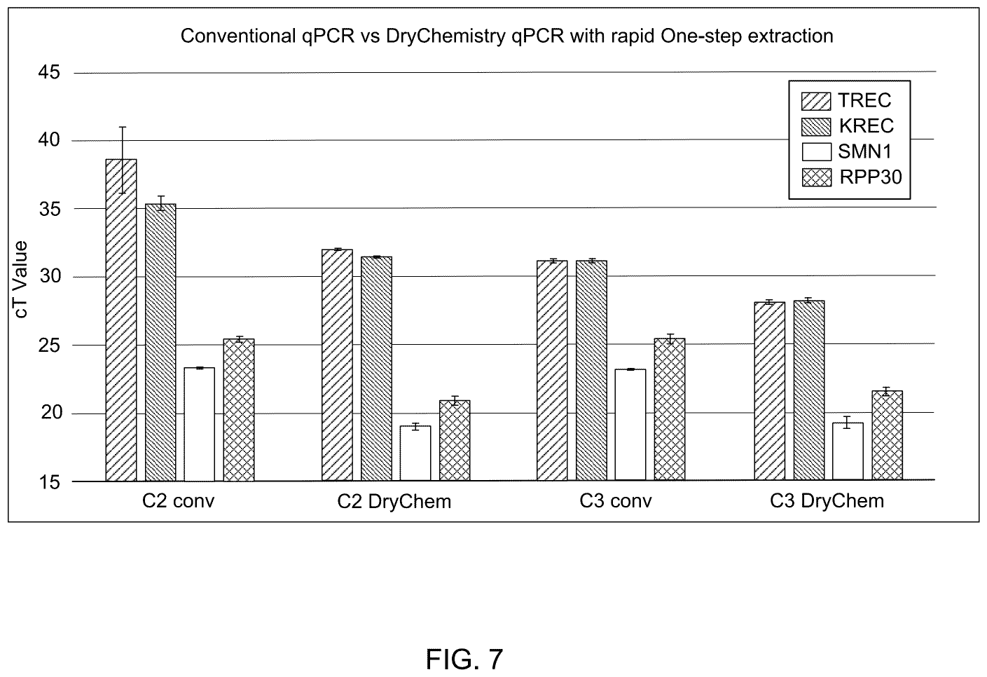

FIG. 6 shows an exemplary workflow. This inhibitor-tolerant dry chemistry qPCR assay comprises qPCR reagents dried in PCR plate wells, which are resuspended with a one-step DNA extract from DB S.

As shown in FIG. 6, in some embodiments, Step 1 included extracting a portion (e.g., a disc) of the sample carrier 606 (e.g., a dried blood spot card) that comprises at least a portion of the dried blood spot 608. In some embodiments, a biological sample puncher may be used for removing the portion of the sample carrier 606 that includes the portion of the dried blood spot 608. In some embodiments, the duration of Step 1 is about 10 minutes.

Step 2 included extracting DNA from the dried blood spot sample. In some embodiments, the DNA extraction protocol consisted of only one step where a DBS sample (e.g., a dried blood spot disc having a diameter of about 3.2 mm) was incubated for about 20 minutes with about 80-160 μl of an elution solution (e.g., about 30 mM Trizma® base and about 20 mM KOH) at about 70° C. while shaking at about 700 rpm.

Step 3 included providing a vessel (e.g., a PCR plate comprising one or more wells) comprising a reaction solution (e.g., qPCR reagents) dried thereon. In some embodiments, all qPCR reagents needed in the assay were air-dried to the bottom of the qPCR plate wells. In some embodiments, the drying procedure only required an oven (e.g., a high-quality oven) where the qPCR reagents were dried for about 30-40 minutes at about 80° C. About 15-20 μl of the crude DNA extract, that was eluted from the DBS sample, was then added to the qPCR plate to resuspend the dried qPCR reagents. Step 4 included performing qPCR on the eluted biological sample contacting the PCR plate and reaction solution after the plate was sealed. Step 5 included performing qPCR analysis.

FIG. 7 shows a four-analyte multiplex qPCR assay with a conventional polymerase and purified DNA as template compared against the dry chemistry qPCR assay with inhibitor-tolerant components and the disclosed one-step DNA extraction. The starting sample material for both protocols was the same, i.e., the same C2 and C3 punches (e.g., sample carriers) were used for both conventional (e.g., wet chemistry) and dry chemistry qPCR assays. The results (i.e., cycle threshold (Ct) values) demonstrated that the dry chemistry qPCR assay had a better performance given that a lower Ct-value denotes a more efficient amplification of targets.

The DBS samples for the wet chemistry were processed with a multistep extraction protocol resulting in a much more purified DNA sample. The dry chemistry DBS samples were extracted according to the disclosed one-step protocol resulting in a crude DNA sample with high concentrations of blood-based inhibitors.

While this specification contains many specific implementation details, these should not be construed as limitations on the scope of any technology or of what may be claimed, but rather as descriptions of features that may be specific to particular embodiments. Certain features that are described in this specification in the context of separate embodiments can also be implemented in combination in a single embodiment in part or in whole. Conversely, various features that are described in the context of a single embodiment can also be implemented in multiple embodiments separately or in any suitable sub-combination. Moreover, although features may be described herein as acting in certain combinations and/or initially claimed as such, one or more features from a claimed combination can in some cases be excised from the combination, and the claimed combination may be directed to a sub-combination or variation of a sub-combination.

Similarly, while operations are depicted in the drawings and the specification in a particular order, this should not be understood as requiring that such operations be performed in the particular order shown or in sequential order, or that all illustrated operations be performed, to achieve desirable results. In certain circumstances, multitasking and parallel processing may be advantageous. Particular embodiments of the subject matter have been described. Other embodiments are within the scope of the claims.

### OTHER EMBODIMENTS

It is to be understood that while the invention has been described in conjunction with the detailed description thereof, the foregoing description is intended to illustrate and not limit the scope of the invention, which is defined by the scope of the appended claims.

Disclosed are systems and kits that can be used for, can be used in conjunction with, can be used in preparation for, or are products of the disclosed methods. These and other systems, kits, and methods are disclosed herein, and it is understood that combinations, subsets, interactions, groups, etc. of these systems, kits, and methods are disclosed. That is, while specific reference to each various individual and collective combinations and permutations of these systems, kits, and methods may not be explicitly disclosed, each is specifically contemplated and described herein. For example, if a particular system, kit, or a particular method is disclosed and discussed and a number of systems, kits, or methods are discussed, each and every combination and permutation of the systems, kits, and the methods are specifically contemplated unless specifically indicated to the contrary. Likewise, any subset or combination of these is also specifically contemplated and disclosed.

Other aspects, advantages, and modifications are within the scope of the following claims.

1.-63. (canceled)

64. A method of analyzing a biological sample, the method comprising:
a) obtaining a sample carrier that comprises the biological sample dried thereon;
b) creating a combination within a vessel, the combination comprising the sample carrier, an elution solution, and a reaction solution, wherein the sample carrier has not been subjected to incubation prior to creating the combination; and
c) incubating the combination within the vessel to elute the biological sample from the sample carrier and to perform qPCR on the biological sample.

65. The method of claim 64, wherein the incubating further comprises incubating at a first temperature to elute the biological sample, and incubating at a second temperature to perform qPCR.

66. The method of claim 65, wherein the first temperature is about 20° C. to about 100° C.

67. The method of claim 66, wherein the combination is incubated within the vessel at the first temperature for about 5 minutes to about 60 minutes.

68. The method of claim 64, wherein the biological sample is blood or saliva.

69. The method of claim 64, wherein the elution solution and the biological sample are not removed from the vessel prior to performing the qPCR.

70. The method of claim 64, wherein the biological sample is collected on a swab from a surface.

71. The method of claim 70, wherein the biological sample collected on the swab is selected from the group consisting of, buccal cells, nasal tissue and/or mucosa, throat tissue and/or mucosa, and combinations thereof.

72. The method of claim 64, wherein a) and b) do not include a washing step.

73. The method of claim 64, wherein, prior to a) the reaction solution is lyophilized in the vessel.

74. The method of claim 73, wherein, prior to a), the vessel comprising a lyophilized reaction solution is stored at a temperature of about −20° C. to about 35° C.

75. The method of claim 64, wherein the reaction solution comprises an inhibitor-resistant polymerase.

76. The method of claim 64, wherein the reaction solution comprises a preservative.

77. The method of claim 64, wherein the reaction solution comprises one or more salts selected from the group consisting of NH₄Cl, KCl, MgCl₂, Mg²⁺, K⁺, and combinations thereof.

78. The method of claim 64, wherein the reaction solution includes an indicator selected from the group consisting of cresol red, bromophenol blue, and xylene cyanol.

79. The method of claim 64, wherein the reaction solution includes a stabilizer selected from the group consisting of trehalose, mannitol, bovine serum albumin (BSA), polyethylene, and combinations thereof.

80. The method of claim 64, wherein the qPCR is performed without a pre-amplification step.

81. A system for analyzing a biological sample dried on a sample carrier, the system comprising:
a vessel;
a liquid handler;
a qPCR system configured to perform qPCR on the biological sample; and
a controller configured to cause the liquid handler to create a combination in the vessel, the combination including an elution solution, a reaction solution, and the biological sample dried on the sample carrier, where the sample carrier has not been subjected to incubation prior to creating the combination.

82. A kit for use in analyzing a biological sample dried on a sample carrier, the kit comprising:
an elution solution;
a reaction solution; and
instructions for performing qPCR on a biological sample dried on a sample carrier, the instructions including:
creating a combination, the combination comprising the sample carrier with the biological sample dried thereon, the reaction solution, and the elution solution, wherein the sample carrier has not been subjected to incubation prior to creating the combination; and
incubating the combination to elute the biological sample from the sample carrier and to perform qPCR on the biological sample,
wherein said instructions do not include instructions for subjecting the sample carrier with the biological sample dried thereon to incubation prior to creating the combination.

83. A method of analyzing a biological sample, the method comprising:
a) obtaining a sample carrier that comprises the biological sample dried thereon;
b) incubating the sample carrier with an elution solution to elute the biological sample from the sample carrier; and
c) contacting a vessel comprising a reaction reagent dried thereon with the eluted biological sample to perform qPCR on the eluted biological sample.
